(19) United States
(12) Patent Application Publication
(10) Pub. No.: US 2024/0281972 A1
(43) Pub. Date: Aug. 22, 2024

(54) X-RAY DIAGNOSTIC APPARATUS, MEDICAL IMAGE PROCESSING APPARATUS, AND MEDICAL IMAGE PROCESSING METHOD

(71) Applicant: Canon Medical Systems Corporation, Otawara-shi (JP)

(72) Inventors: Jumpei OGASAWARA, Nasushiobara (JP); Hisayuki UEHARA, Otawara (JP); Hajime YOSHIDA, Nasushiobara (JP); Yasuto HAYATSU, Otawara (JP); Toshiya WAKU, Yaita (JP); Naotaka SATO, Otawara (JP)

(73) Assignee: Canon Medical Systems Corporation, Otawara-shi (JP)

(21) Appl. No.: 18/437,078

(22) Filed: Feb. 8, 2024

(30) Foreign Application Priority Data
Feb. 16, 2023 (JP) 2023-022665

(51) Int. Cl. G06T 7/00; A61B 6/00; G06V 10/70; G06V 10/774; G06V 10/776

(52) U.S. Cl. CPC G06T 7/0014; A61B 6/5211; G06V 10/774; G06V 10/776; G06V 10/87; G06T 2207/10116; G06T 2207/20081; G06T 2207/20084; G06T 2207/30004; G06V 2201/03

(57) ABSTRACT

According to one embodiment, an X-ray diagnostic apparatus includes processing circuitry. The processing circuitry is configured to acquire an X-ray image. The processing circuitry is configured to perform a determination as to whether or not a first trained model, to which the X-ray image is to be input, is adapted to the X-ray image, and if the determination indicates that the first trained model is not adapted to the X-ray image, apply image processing to the X-ray image so that the first trained model serves as an adapted model.

### CROSS-REFERENCE TO RELATED APPLICATION(S)

This application is based upon and claims the benefit of priority from prior Japanese Patent Application No. 2023-022665, filed Feb. 16, 2023, the entire contents of which are incorporated herein by reference.

### FIELD

Embodiments described herein relate generally to an X-ray diagnostic apparatus, a medical image processing apparatus, and a medical image processing method.

### BACKGROUND

An X-ray diagnostic apparatus which processes images using a machine learning-based trained model has been known in recent years. A trained model of this type applies, for example, image processing such as noise reduction or lesion part segmentation to an input X-ray image, and outputs the X-ray image after the image processing. Such a trained model can show excellent performance and return a desired image processing result if the input X-ray image falls within the scope of the training data, that is, if the trained model is well adapted to the X-ray image. However, if the input X-ray image falls outside the scope of the training data, that is, if the trained model is not adapted to the X-ray image, the trained model cannot exhibit its performance and might output an unintended image processing result. For example, supposing that an input X-ray image involves an X-ray condition or a patient condition that falls outside the scope of the training data, the trained model could output an X-ray image with a brightness changed and artifacts created. Circumstances with such an unadapted trained model, which would likely output an unintended image processing result, could incur occurrence of an erroneous diagnosis, a need to redo imaging operations, an unnecessary exposure to radiation, and so on.

A trained model is therefore required to be adapted to X-ray images to be input, so that it can exhibit its performance and provide a desired image processing result. Meanwhile, since whether or not the performance of a trained model would be exhibited is not detectable unless the adaptation or non-adaptation to X-ray images is taken into account, a user is not able to know whether or not a trained model would exhibit its performance.

### BRIEF DESCRIPTION OF THE DRAWINGS

FIG. 1 is a block diagram showing a configuration of an X-ray diagnostic apparatus according to a first embodiment.

FIG. 2 is a schematic diagram for explaining a first trained model according to the first embodiment.

FIG. 3 is a schematic diagram for explaining training information for the first trained model according to the first embodiment.

FIG. 4 is a schematic diagram for explaining image processing (preprocessing) according to the first embodiment.

FIG. 5 is a flowchart for explaining operations in the first embodiment.

FIG. 6 is a schematic diagram for explaining operations of step ST31 in the first embodiment.

FIG. 7 is a block diagram showing a configuration of an X-ray diagnostic apparatus according to a second embodiment.

FIG. 8 is a flowchart for explaining operations in the second embodiment.

FIG. 9 is a flowchart for explaining operations in a third embodiment.

FIG. 10 is a block diagram showing a configuration of an X-ray diagnostic apparatus according to a fourth embodiment.

FIG. 11 is a flowchart for explaining operations in the fourth embodiment.

FIG. 12 is a flowchart for explaining operations in a fifth embodiment.

FIG. 13 is a flowchart for explaining operations in a sixth embodiment.

FIG. 14 is a schematic diagram for explaining a second trained model according to a seventh embodiment.

FIG. 15 is a flowchart for explaining operations in the seventh embodiment.

FIG. 16 is a block diagram showing a configuration of a medical image processing apparatus according to an eighth embodiment.

### DETAILED DESCRIPTION

In general, according to one embodiment, an X-ray diagnostic apparatus includes processing circuitry. The processing circuitry is configured to acquire an X-ray image. The processing circuitry is configured to perform a determination as to whether or not a first trained model, to which the X-ray image is to be input, is adapted to the X-ray image, and if the determination indicates that the first trained model is not adapted to the X-ray image, apply image processing to the X-ray image so that the first trained model serves as an adapted model.

Each embodiment will be described with reference to the drawings. The description will use the same reference numerals for the structural features or components having the same or substantially the same functions and configurations, so as to omit redundant descriptions.

### First Embodiment

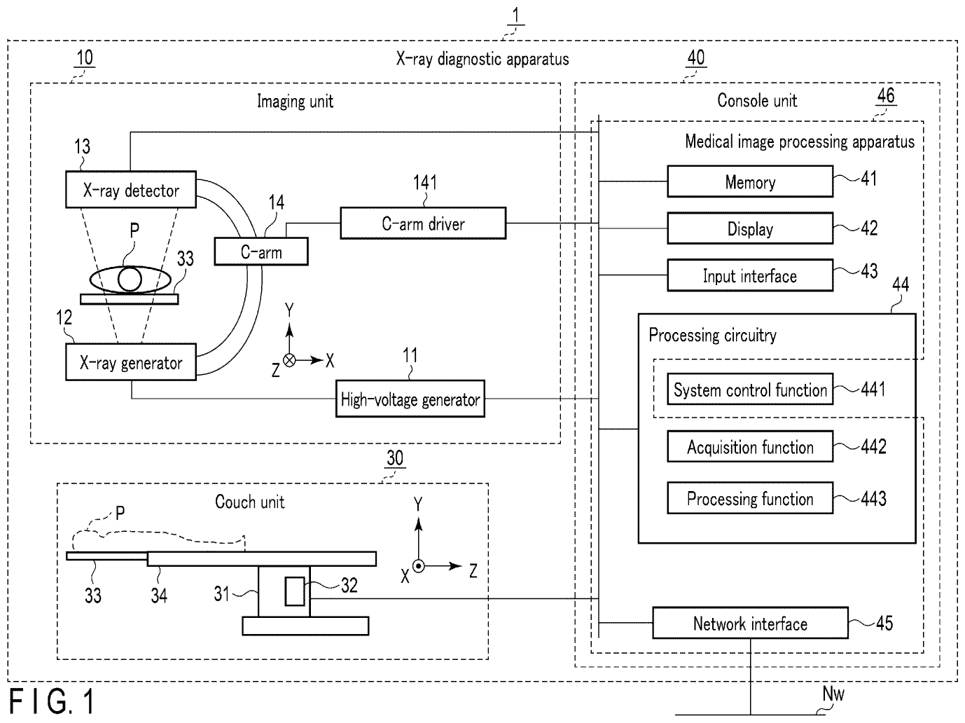

FIG. 1 is a block diagram showing a configuration of an X-ray diagnostic apparatus according to the first embodiment. This X-ray diagnostic apparatus 1 may be, for example, an apparatus for cardiovascular use. By way of example, the description will assume the X-ray diagnostic apparatus 1 to be of a single plane structure, but no limitation is intended by this. The X-ray diagnostic apparatus 1 may additionally or instead adopt a bi-plane structure.

More specifically, in one example, the X-ray diagnostic apparatus 1 includes an imaging unit 10, a couch unit 30, and a console unit 40. The imaging unit 10 includes a high-voltage generator 11, an X-ray generator 12, an X-ray detector 13, a C-arm 14, and a C-arm driver 141.

The high-voltage generator 11 generates and outputs high voltages to an X-ray tube so that the high voltages are applied between an anode and a cathode of the X-ray tube in order to accelerate thermal electrons produced from the cathode.

The X-ray generator 12 causes its components to generate X-rays. More specifically, the X-ray generator 12 is provided with the X-ray tube for radiating X-rays toward a subject P, and an X-ray diaphragm which has functions of delimiting the irradiation field of the X-rays, attenuating the X-rays for a part of the irradiation fields, and so on.

The X-ray tube generates X-rays. More specifically, the X-ray tube is a vacuum tube having a cathode for producing thermal electrons and an anode for generating X-rays by receiving the thermal electrons flying from the cathode. Examples of the X-ray tube include an X-ray tube of a rotating anode type, which generates X-rays by emitting thermal electrons to a rotating anode. The X-ray tube is connected to the high-voltage generator 11 through a high-voltage cable. The high-voltage generator 11 applies a tube voltage between the cathode and the anode. Upon application of this tube voltage, thermal electrons depart from the cathode toward the anode. As the thermal electrons fly from the cathode toward the anode, a tube current flows. Thus, with the application of high voltage and the supply of filament current from the high-voltage generator 11, thermal electrons fly from the cathode to anode and collide with the cathode, thereby generating X-rays.

The X-ray diaphragm is arranged between the X-ray tube and the X-ray detector 13. The X-ray diaphragm typically employs diaphragm blades, as well as an added filter and a compensating filter. The X-ray diaphragm limits the X-rays generated by the X-ray tube by blocking the X-ray paths excluding the area of opening so that the X-rays will be applied to only the region of interest of the subject P. In one example, the X-ray diaphragm includes four diaphragm blades each constituted by a plate made of lead, and slides these diaphragm blades to adjust the X-ray shield area into a desired size. The diaphragm blades of the X-ray diaphragm may be driven by a driver (not shown in the figure) according to the region of interest input by an operator via a later-described input interface 43. The X-ray diaphragm also has a slit which can receive insertion of an added filter for adjusting the total filtration of X-rays. The X-ray diaphragm further has an accessory slot which can receive insertion of a lead mask or a compensating filter for use during X-ray examination operations. The compensating filter may include an ROI (region of interest) filter having a function of attenuating or reducing the amount of X-ray radiation.

The X-ray detector 13 detects X-rays transmitted through the subject P. This X-ray detector 13 may be a type that converts X-rays directly into electric charges, or a type that first converts X-rays into light and then converts the light into electric charges. The description will assume the former type, but the X-ray detector 13 may also be the latter type. Specifically, and for example, the X-ray detector 13 includes a planar flat panel detector (FPD) for converting the X-rays transmitted through the subject P into electric charges and accumulating these electric charges, and a gate driver for generating drive pulses for reading the electric charges accumulated in the FPD. The FPD includes micro-sensor elements two-dimensionally arranged in the column direction and the line direction. The sensor elements each include a photoelectric film, a charge accumulation capacitor, and a thin film transistor (TFT). The photoelectric film senses X-rays and generates electric charges according to the amount of incident X-rays. The charge accumulation capacitor accumulates the electric charges generated at the photoelectric film. The TFT outputs, at predetermined timings, the electric charges accumulated at the charge accumulation capacitor. The accumulated electric charges are sequentially read out with the drive pulses supplied from the gate driver.

While not illustrated in the figures, there are projection data generation circuitry and projection data storage circuitry arranged subsequently to the X-ray detector 13. The projection data generation circuitry includes a charge-voltage converter, an analog-digital (A/D) converter, and a parallel-serial converter. The charge-voltage converter converts the electric charges read from the FPD in units of rows or columns in a parallel manner into voltages. The A/D converter converts the output of this charge-voltage converter into digital signals. The parallel-serial converter converts the digitally converted parallel signals into time-series serial signals. The projection data generation circuitry supplies these serial signals to the projection data storage circuitry as time-series projection data. The projection data storage circuitry sequentially stores the time-series projection data supplied from the projection data generation circuitry to generate two-dimensional projection data (X-ray images). In other words, the X-ray detector 13 detects X-rays transmitted through the subject and sequentially generates X-ray images. The X-ray images (two-dimensional projection data) are then stored in a memory 41.

The C-arm 14 holds the X-ray generator 12 and the X-ray detector 13 in such an arrangement that they face each other with the subject P and a couch top 33 located therebetween, so that X-ray imaging of the subject P on the couch top 33 is enabled. By way of example, the following description will assume the C-arm 14 to be a type that is suspended from the ceiling, but this is not a limitation. The C-arm 14 may be, for example, a floor-mounted type.

Specifically, the C-arm 14 is held by a holding portion (not shown in the figure) so that it is rotatable about an axis extending in an X direction orthogonal to both a Y direction perpendicular to the couch top 33 and a Z direction along the long axis of the couch top 33. The C-arm 14 is of a substantially arc shape, which is concentric on the Z-direction axis, and held by the holding portion so that it is further slidable along the substantially arc shape. That is, the C-arm 14 is also capable of the sliding movement about the Z-direction axis. The C-arm 14, with the capability of making said rotational movement about the X-direction axis through the holding portion (“main rotational movement”) in combination with this sliding movement, can enable X-ray image observations at various angles and from various directions. The C-arm 14 may further be rotatable about the Y-direction axis, whereby the center of the sliding movement coincides with, for example, the X-direction axis. Note that the focal point of the X-rays from the X-ray generator 12 and the imaging axis extending through the center of the detection plane of the X-ray detector 13 are designed to intersect each other at a single point on the axis serving as the center of the sliding movement and also on the axis serving as the center of the main rotational movement. Such a point of intersection is generally called an “isocenter”. An isocenter is not displaced with the sliding movement or the main rotational movement of the C-arm 14. As such, once a concerned site is positioned at the isocenter, observation of the site through medical moving images acquired from the slicing movement or the main rotational movement of the C-arm 14 is facilitated.

For the C-arm 14, multiple power sources may be provided at suitable locations in order to realize such sliding and rotational movements. These power sources constitute the C-arm driver 141. The C-arm driver 141 reads drive signals from a later-described system control function 441 to cause the C-arm 14 to make its sliding movement, rotational movement, linear movement, etc. The C-arm 14 is also provided with one or more state detectors (not shown in the figure) for detecting respective information on an angle or orientation, a position, etc. of the C-arm 14. The state detectors each include, for example, a potentiometer for detecting a rotation angle, a movement amount, etc., an encoder which is a position sensor, and so on. Examples of the available encoder include a so-called absolute encoder of a magnetic type, a brush type, a photoelectric type, or the like. As the state detectors, various position detecting mechanisms may be discretionarily adopted, such as a rotary encoder outputting a rotational displacement in the form of digital signals, a linear encoder outputting a linear displacement in the form of digital signals, and so on.

The couch unit 30 is a unit which movably carries the subject P placed on itself and includes a base 31, a couch driver 32, the aforementioned couch top 33, and a support frame 34.

The base 31 is a housing furnished on the floor and supporting the support frame 34 in such a manner that the support frame 54 can move vertically (in the Y direction).

The couch driver 32 may be disposed in the housing of the couch unit 30 and be constituted by an actuator for moving the couch top 33, on which the subject P is placed, in the longitudinal direction of the couch top 33 (in the Z direction). The couch driver 32 reads drive signals from the system control function 441 to cause the couch top 33 to move horizontally and vertically with respect to the floor face. Movement of the C-arm 14 or the couch top 33 varies the positional relationship of the imaging axis to the subject P. Note that the couch driver 32 may move not only the couch top 33 but also the support frame 34 in the longitudinal direction of the couch top 33.

The couch top 33 is set on the upper side of the support frame 34 and may be a plate for the placement of the subject P.

The support frame 34 is provided at the upper portion of the base 31 and supports the couch top 33 so that the couch top 33 can slide in its longitudinal direction.

The console unit 40 includes the aforementioned memory 41 and input interface 43, and also a display 42, processing circuitry 44, and a network interface 45.

The memory 41 includes a memory main part for storing electric information, such as a read only memory (ROM), a random access memory (RAM), a hard disk drive (HDD), and/or an image memory, and peripheral circuitry pertaining to the memory main part, such as a memory controller and/or a memory interface. The memory 41 stores, for example, one or more programs for execution by the processing circuitry 44, X-ray images generated by the processing circuitry 44, data for use in the processing by the processing circuitry 44, data under processing, data after processing, and so on. The memory 41 also stores one or more trained models, training information, preprocessing information, etc. for use in the processing by the processing circuitry 44. The training information contains the center of gravity and a standard deviation of the distribution of first feature amounts of the training data for a first trained model. The memory 41 is one example of a storage for storing the center of gravity and a standard deviation of the distribution of the first feature amounts.

The display 42 includes a display main part for displaying medical images, etc., internal circuitry for supplying display signals to the display main part, and peripheral circuitry including connectors, cables, or the like for connecting the display main part and the internal circuitry, etc. The internal circuitry generates display data by superimposing supplemental information, such as subject information and projection data generation conditions, on the image data received from the processing circuitry 44, and subjects this display data to D/A conversion and TV format conversion for display through the display main part.

The input interface 43 allows an input of subject information, setting of X-ray imaging conditions including X-ray irradiation conditions, an input of various command signals, and so on. The input interface 43 is realized by components for providing, for example, instructions for movement of the C-arm 14, setting of a region of interest (ROI), etc., and such components include a trackball, switch buttons, a mouse, a keyboard, a touch pad which allows input operations through contact of the operation screen, and a touch panel display which integrates a display screen and a touch pad. The input interface 43 is connected to the processing circuitry 44, and converts input operations received from an operator into electric signals and outputs the electric signals to the processing circuitry 44. Note that, in the disclosure herein, the input interface 43 is not limited to physical operation components such as a mouse, a keyboard, or the like. Examples of the input interface 43 also include processing circuitry for electric signals, which receives an electric signal corresponding to an input operation from an external input device separate from the apparatus unit and outputs this electric signal to the processing circuitry 44.

The processing circuitry 44 is a processor to read and execute a program or programs in the memory 41 for realizing corresponding functions including the aforementioned system control function 441, an acquisition function 442, and a processing function 443. Programs available as such a program include, for example, a medical image processing program for causing a computer (e.g., a medical image processing apparatus 46) to realize the acquisition function 442 and the processing function 443. This medical image processing program may further cause the computer to realize at least one of an estimation function and/or a preparation function described later as appropriate. While FIG. 1 assumes that the processing circuitry 44 is a single circuitry element for realizing each of the system control function 441, the acquisition function 442, and the processing function 443, the processing circuitry 44 may be constituted by a combination of multiple independent processors running programs to realize the respective functions. Note also that the system control function 441, the acquisition function 442, and the processing function 443 may be called a system control circuit, an acquisition circuit, and a processing circuit, respectively, and they may be implemented as individual hardware circuits. These explanations about programs, processors, and hardware circuits are applicable also to the estimation function and the preparation function described later. The processing circuitry 44 is one example of an acquirer, a processor, an estimator, and a preparator.

In one example, the system control function 441 handles information, such as command signals and various initial settings and conditions input by an operator via the input interface 43, in such a manner that it temporarily holds the information and then sends the information to each of the corresponding processing functions of the processing circuitry 44. Also in one example, the system control function 441 controls the C-arm driver 141 and the couch driver 32 using information input via the input interface 43 for the driving of the C-arm 14 and the couch top 33. In one example, the system control function 441 reads stored information on various initial settings and conditions, etc., and controls the high-voltage generator 11 for the X-ray irradiation conditions including values of the tube current, the tube voltage, the irradiation time, and so on.

The acquisition function 442 acquires one or more X-ray images from the memory 41. In one example, the acquisition function 442 acquires an X-ray image in the memory 41 once the X-ray images sequentially generated by the X-ray detector 13 are stored in the memory 41. The acquisition function 442 may include a function for setting collection conditions for an X-ray image to acquire.

The processing function 443 determines whether or not the first trained model, to which an X-ray image acquired by the acquisition function 442 is to be input, is adapted to such an X-ray image, and if not, applies image processing to the X-ray image so that the first trained model will serve as an adapted model. For example, if it is found that the first trained model is not adapted due to the acquired X-ray image having a brightness that falls outside the scope of the training data, the processing function 443 performs image processing according to the reason of non-adaptation, e.g., applying image processing for brightness adjustment to the X-ray image. A background of the reason for non-adaptation is that a feature amount that largely deviates from the scope of the training data for the trained model prevents the trained model from exhibiting its performance. Accordingly, the processing function 443 corrects the X-ray image into an X-ray image for good adaptation by applying image processing (preprocessing) to the X-ray image. This allows the first trained model to exhibit the performance for the corrected X-ray image.

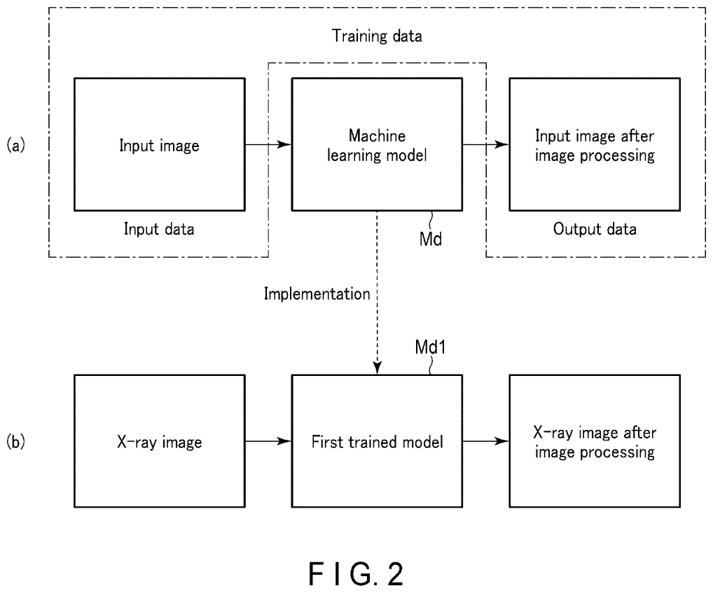

The first trained model is an already-trained model Md1 which has been obtained by having a machine learning model Md undergo a machine learning process with training data including input data and output data and which is set in the memory 41. See FIG. 2(a) illustrating a training instance. The first trained model Md1 corresponds to the trained machine learning model Md in implementation, and upon receiving an input of the acquired X-ray image, it outputs the X-ray image to which a given image processing has been applied. See FIG. 2(b) illustrating a use instance. Note that, for implementation, the first trained model Md1 here may be set in the memory 41 in advance, before shipment of the X-ray diagnostic apparatus 1 from the factory. As another option, the trained machine learning model Md may be acquired from a server device or the like (not shown in the figure) and installed in the memory 41 for implementation of the first trained model Md1, after the factory shipment of the X-ray diagnostic apparatus 1. These are also true of each embodiment and modification set forth below.

The machine learning process utilizes, for example, a deep neural network (DNN) which is a multi-layered neural network intended for deep learning. As the DNN, for example, a recurrent neural network (RNN) may be used for moving images, and a convolutional neural network (CNN) may be used for still images. This type of a CNN may be called a “deep CNN (DCNN)”. The embodiment assumes the use of a DCNN as the trained model. An RNN may include a long short-term memory (LSTM). Note, however, the machine learning process is not limited to the utilization of a DNN, but it may also utilize, for example, supervised machine learning algorithms such as a support vector machine (SVM). That is, the machine learning process may adopt supervised learning algorithms where to-be-processed images constitute input data and processed images constitute output data. The foregoing explanation about machine learning will likewise apply to all the machine learning models Md and the first trained model Md1, and also to a later-described second trained model.

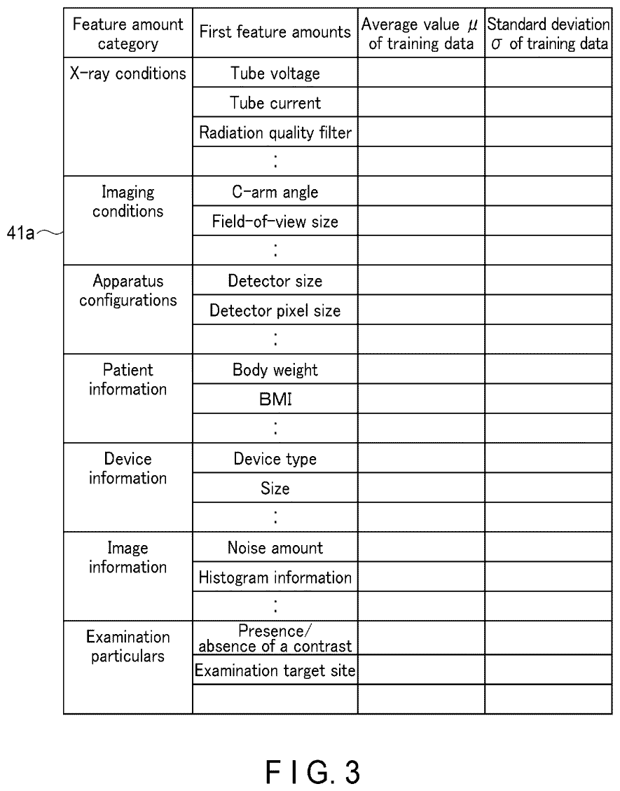

The training data here includes a set of input data and output data, where the input data is an input image which is an X-ray image before image processing, and the output data is the input image after the image processing. The machine learning process employs multiple pieces of training data. For such multiple training data pieces, information indicative of a distribution of the training data as shown in FIG. 3 is stored as training information 41a in the memory 41. The training information 41a contains a feature amount category, the aforementioned first feature amounts, an average value μ, and a standard deviation σ. The feature amount category refers to items categorizing the first feature amounts, which are feature amounts of the input data (X-ray images) included in the training data for the first trained model Md1. Examples of items that may be discretionarily adopted as the feature amount category are X-ray conditions, imaging conditions, apparatus configurations, patient information, device information, image information, and examination particulars. Here, the X-ray conditions are items to which the first feature amounts related to X-rays belong, and include a tube voltage, a tube current, a radiation quality filter, etc. The imaging conditions are items to which the first feature amounts related to an imaging operation belong, and include a C-arm angle, a field-of-view size, etc. The apparatus configurations are items to which the first feature amounts related to configurations of an imaging apparatus belong, and include a detector size, a detector pixel size, etc. The patient information is items to which the first feature amounts related to a patient belong, and includes a body weight, a body-mass index (BMI), etc. The device information is items to which the first feature amounts related to a device belong, and includes a type, a size, etc. of a device such as a catheter, a stent, or the like appearing in X-ray images. The image information is items to which the first feature amounts indicating image feature amounts belong, and includes a noise amount, histogram information, etc. The examination particulars are items to which the first feature amounts related to image contents corresponding to an examination belong, and include a presence/absence of a contrast, an examination target site, etc. The first feature amounts are feature amounts of the training data for the first trained model Md1. The average value μ is calculated for each item of the first feature amounts of the training data and constitutes one example of data indicative of a distribution of the first feature amounts. More specifically, the average value μ indicates an average of the first feature amounts of the training data and corresponds to the center of gravity of the distribution of the first feature amounts. The standard deviation σ is calculated for each item of the first feature amounts of the training data and constitutes another example of data indicative of a distribution of the first feature amounts. More specifically, the standard deviation σ is a value indicating a standard deviation of the first feature amounts of the training data and corresponds to the size of a standardly deviating distance from the center of gravity of the distribution of the first feature amounts.

The processing function 443 may consider at what degree a second feature amount of the acquired X-ray image deviates from the center of gravity of the distribution of the first feature amounts of the training data for the first trained model Md1, and perform the aforementioned determination as to whether or not the first trained model is adapted to the X-ray image according to this degree of deviation. More specifically, and for example, the processing function 443 may acquire the degree of deviation by dividing a difference between the second feature amount and the center of gravity by the standard deviation, and perform the determination according to whether or not this degree of deviation is equal to or below a threshold.

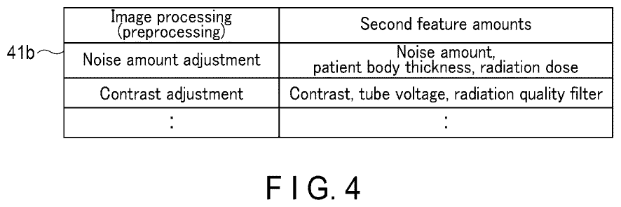

The processing function 443 may, if the result of the determination is “No”, select one or more second feature amounts having a degree of deviation greater than the threshold and conduct image processing associated with the selected second feature amount or amounts. Here, the processing function 443 may read, from the memory 41, preprocessing information 41b in which contents of image processing to be performed as preprocessing are associated with second feature amounts of the X-ray image as shown in FIG. 4, and specify the image processing associated with the selected second feature amount based on the preprocessing information 41b. In FIG. 4, noise amount adjustment as an image processing content is associated with a noise amount, a patient body thickness, and a radiation dose which represent the second feature amounts. Similarly, contrast adjustment as an image processing content is associated with a contrast, a tube voltage, and a radiation quality filter which represent the second feature amounts. The preprocessing information 41b may further associate an imaging purpose with the image processing contents and the second feature amounts. More specifically, and for example, if the imaging purpose is to check a stent, this imaging purpose is associated with image processing of adjusting an edge enhancement filter and the second feature amount including spatial resolution. Also, the processing function 443 may set an average value of the distribution of image feature amounts among the first feature amounts to be a target value of the image processing, and apply the image processing to the X-ray image based on this target value. The image feature amounts for this setting are the first feature amounts related to the image information in the training information 41a. That is, the average value of the distribution of image feature amounts refers to an average value of the training data having the first feature amounts related to the image information in the training information 41a.

The network interface 45 is circuitry for connecting the console unit 40 to a network Nw for communication with other apparatuses and entities. As the network interface 45, for example, a network interface card (NIC) may be employed. In the following disclosure, such a description as the network interface 45 being involved in communications with other apparatuses, etc. will be omitted.

The memory 41, the display 42, the input interface 43, and the processing circuitry 44 with the acquisition function 442 and the processing function 443 together constitute a medical image processing apparatus 46 which performs a medical image processing method. The medical image processing apparatus 46 may further include the estimation function and the preparation function described later.

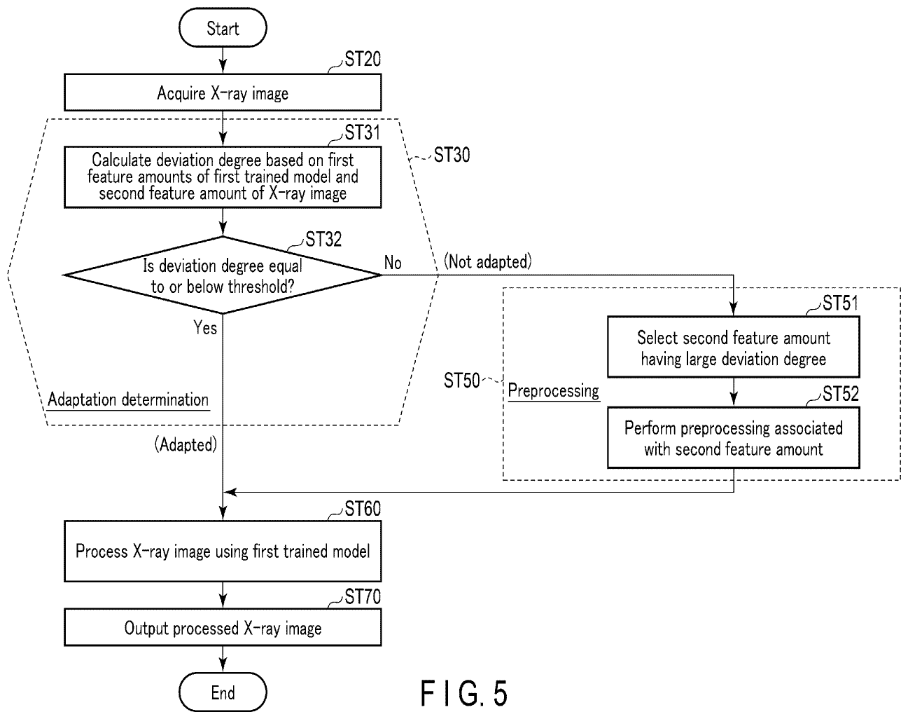

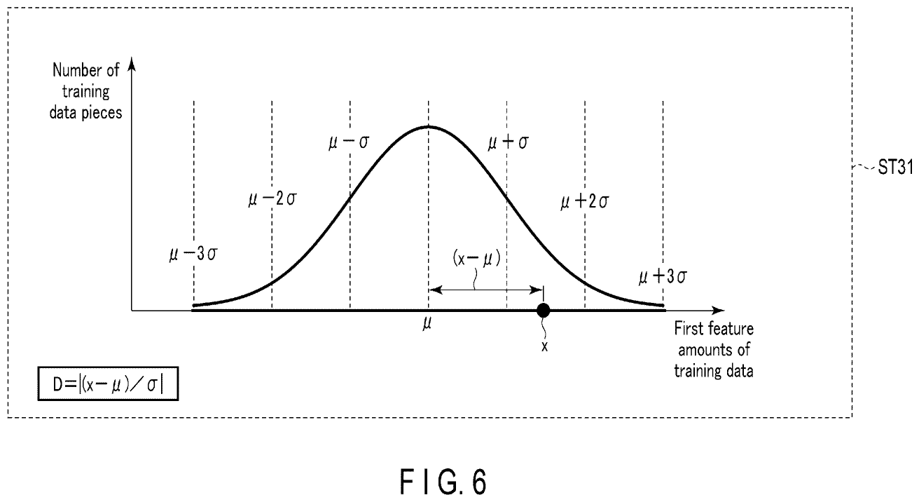

Next, operations of the X-ray diagnostic apparatus 1 configured as above will be described with reference to the flowchart in FIG. 5 and the schematic diagram in FIG. 6.

First, in response to an operator operating the input interface 43, the processing circuitry 44 of the X-ray diagnostic apparatus 1 sets collection conditions for an X-ray image and controls the imaging unit 10 according to the collection conditions so as to perform X-ray imaging.

In step ST20, the X-ray generator 2 generates X-rays, and the X-ray detector 13 detects the X-rays transmitted through a subject P and sequentially generates X-ray images. The sequentially generated X-ray images from the X-ray detector 3 are stored in the memory 41. The processing circuitry 44 accordingly acquires one or more X-ray images.

In step ST30, the processing circuitry 44 determines whether or not the first trained model Md1, to which the acquired X-ray image is to be input, is adapted to the X-ray image. This step ST30 for adaptation determination is constituted by steps ST31 to ST32.

In step ST31, for example, the processing circuitry 44 calculates a degree of deviation based on the first feature amounts of the training data for the first trained model Md1 and the second feature amount of the acquired X-ray image. A degree of deviation is an index indicating to what extent the second feature amount deviates from the center of gravity (average value μ) of the distribution of the first feature amounts. In one example, a deviation degree D is acquired by dividing a difference between the second feature amount x and the average value μ of the first feature amounts by the standard deviation σ of the first feature amounts as shown in FIG. 6 and the following expression (1). Note that the expression (1) may instead include division of an absolute value of the difference by the standard deviation σ (D=|x−μ|/σ). Also note that, since the feature amounts differ in dimensions from each other, the deviation degree D uses the division by the standard deviation σ for the sake of non-dimensional alignment.

$$D = \vert ( x - μ ) / σ \vert \qquad ( 1 )$$

In FIG. 6, the vertical axis represents the number of pieces of the training data, and the horizontal axis represents the first feature amounts of the training data. The curve in FIG. 6 represents the distribution of the first feature amounts having the average value μ and the standard deviation σ.

Subsequently, in step ST32, the processing circuitry 44 performs the determination according to the acquired deviation degree D. In one example, the processing circuitry 44 determines the adaptation or non-adaptation according to whether or not the deviation degree is equal to or below a threshold. As the threshold, for example, a value equal to 3σ of the distribution of the first feature amounts may be used. If the deviation degree D exceeds the threshold, and thus, the determination result is “No”, the operation flow transitions to step ST50. If the deviation degree D is equal to or below the threshold, and thus, the determination result is that the first trained model Md1 is adapted, the operation flow transitions to step ST60. In this manner, step ST30 constituted by steps ST31 to ST32 is finished.

In the event that the operation flow transitions from step ST30 to step ST50, the X-ray image to which the first trained model Md1 is not adapted undergoes image processing (preprocessing) so that the first trained model Md1 will serve as an adapted model. This image processing (preprocessing) in step ST50 is performed as steps ST51 to ST52. In step ST51, the processing circuitry 44 selects the second feature amount of which deviation degree D is greater than the threshold. In step ST52, the processing circuitry 44 performs image processing associated with the selected second feature amount. In one example, the processing circuitry 44 specifies, based on the preprocessing information 41b, the image processing associated with the selected second feature amount. For example, in instances where the patient has a large body thickness or the dose of radiation used is small, which has introduced an increased amount of noise in the X-ray image, noise amount adjustment is specified as the image processing associated with the second feature amount related to a body thickness or a radiation dose. Also for example, in instances where a contrast, a tube voltage, or a radiation quality filter is abnormal, which has caused a contrast reduction of the X-ray image, contrast adjustment is specified as the image processing associated with the second feature amount related to a contrast, a tube voltage, or a radiation quality filter. Then, the processing circuitry 44 sets the average value μ of the distribution of image feature amounts among the first feature amounts to be the target value of this image processing, and applies the image processing to the X-ray image based on the target value. Here, the image processing as the preprocessing is preferably provided with an intensity adjustment capacity so that an intensity of the image processing is adjusted according to the size of the target value. For example, if the target value is a positive value, an X-ray image with an image feature amount being an SD value has a greater noise amount than that in the training data, and therefore, image processing for reducing noise is performed as the preprocessing. On the other hand, if the target value is a negative value, the amount of noise is smaller, and therefore, image processing for adding noise is performed as the preprocessing. In this manner, step ST50 constituted by steps ST51 to ST52 is finished. Upon finishing step ST50, the operation flow transitions to step ST60.

In step ST60, the processing circuitry 44 inputs the X-ray image having an image feature amount close to that of the intended input image to the first trained model Md1, trained to receive such an input image and generate the input image applied with a given image processing, so as to generate the X-ray image after the given image processing.

In step ST70, the processing circuitry 44 causes the display 42 to display, as an outputting operation, the image-processed X-ray image obtained in step ST60.

According to the first embodiment as described above, the processing circuitry 44 acquires an X-ray image. The processing circuitry 44 then determines whether or not the first trained model Md1, to which the X-ray image is to be input, is adapted to the X-ray image, and if not, applies image processing to the X-ray image so that the first trained model Md1 will serve as an adapted model. Thus, in the instance where the first trained model Md1 is not adapted to an acquired X-ray image, this X-ray image is subjected to image processing so that the first trained model Md1 will serve as an adapted model, and therefore, its adaptation to the X-ray image can be secured.

Also according to the first embodiment, the processing circuitry 44 performs the above determination based on a degree of deviation of a second feature amount of the acquired X-ray image from the center of gravity of the distribution of first feature amounts of the training data for the first trained model Md1. There is a property whereby the performance of the trained model for an X-ray image depends on whether or not the X-ray image falls within the scope of the training data. Here, by utilizing this property, whether or not the first trained model is adapted to the acquired X-ray image can be determined based on the degree of deviation of the X-ray image from the training data for the first trained model. Therefore, the above determination can be quantitatively performed using such a degree of deviation as an index.

According to the first embodiment, further, the memory 41 stores, as the training information 41a, the center of gravity and standard deviation of the distribution of the first feature amounts. The processing circuitry 44 acquires the degree of deviation by dividing a difference between the second feature amount and the center of gravity by the standard deviation, and performs the above determination according to whether or not the degree of deviation is equal to or below a threshold. Here, the significance of the difference between the center of gravity (average value μ) of the distribution of the first feature amounts and the second feature amount can be statistically evaluated by using the degree of deviation, which expresses this significance in terms of a multiple of the standard deviation σ. For example, the threshold for the deviation degree may be set to 3σ of the distribution of the first feature amounts so that a statistically abnormal deviation degree can be detected. As such, with the configuration of acquiring a deviation degree based on statistical values and comparing it with a threshold, the determination can be provided along with a statistical evaluation of the deviation degree.

According to the first embodiment, the processing circuitry 44, if the result of the above determination is “No”, selects one or more second feature amounts having a deviation degree greater than the threshold and conducts image processing associated with the selected second feature amount or amounts. As such, with the configuration of subjecting the X-ray image to image processing associated with a largely deviating second feature amount, the effect of performing image processing for establishing the adaptation of the first trained model Md1 to the X-ray image can be further enhanced as compared to cases of performing image processing associated with a second feature amount having a small deviation degree.

According to the first embodiment, the processing circuitry 44 sets an average value of the distribution of image feature amounts among the first feature amounts to be a target value of the image processing, and applies the image processing to the acquired X-ray image based on the target value. As such, with the configuration of subjecting the X-ray image to image processing for approximating to the average training data, the effect of performing image processing for establishing the adaptation of the first trained model Md1 to the X-ray image can be further enhanced as compared to cases of performing image processing based on another target value.

### (Modifications of First Embodiment)

A description will be given of modifications of the first embodiment. The description will basically concentrate on portions constituting the difference, while omitting portions overlapping the first embodiment. Remaining embodiments, modifications, etc. will be set forth in the same manner. Note that each modification of the first embodiment is applicable to each of the other embodiments and their modifications.

### (First Modification)

For the first embodiment, the description has assumed calculation of a single deviation degree in step ST31, but the embodiment is not limited to this. For example, as a deviation degree to acquire, an average value of deviation degrees calculated for the respective sets of the distribution of first feature amounts and a second feature amount may be used. In averaging the deviation degrees, importance levels may be set to the second feature amounts to be improved through image processing by the first trained model Md1 and the weighted average of the deviation degrees may be calculated according to the importance levels. Thus, the acquisition of a deviation degree may be based on calculation of an average value or a weighted average of multiple deviation degrees. According to such a modification, the deviation degree for use in the determination can be acquired based on many sets of the distribution of first feature amounts and a second feature amount, and therefore, a more reliable deviation degree can be acquired.

Also for the first embodiment, the description has assumed acquisition of a deviation degree and performing of the determination for a single feature amount in steps ST31 to ST32, but the embodiment is not limited to this. For example, if a particular combination of feature amounts forms a value of an influential deviation degree, or if such a situation is expected, this combination may be used as the feature amount. As one example, a combination of patient body shape information and C-arm angle information corresponds to X-ray transmittance length information. Since an image varies in brightness, a noise amount, etc., depending on the X-ray transmittance length, such a combination is considered to be a matter of importance in the instances of conducting noise reduction or the like through the machine learning technology. Thus, by employing such a combination as the feature amount, a more accurate determination is enabled.

### Second Embodiment

The second embodiment may be understood as a modification of the first embodiment. The second embodiment adopts a configuration in which, if the first trained model Md1 is not adapted to an acquired X-ray image, image processing (preprocessing) is applied to the X-ray image, and concurrently, collection conditions for the subsequent operations are estimated so that X-ray images to which the first trained model Md1 is adapted will be acquired.

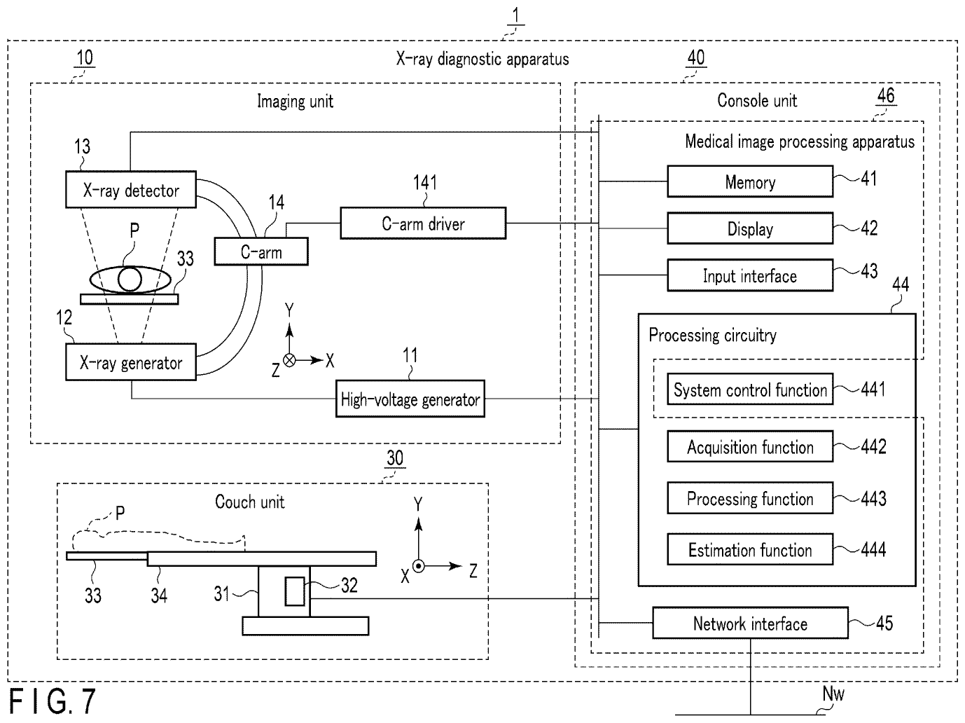

Accordingly, the processing circuitry 44 further realizes, as shown in FIG. 7, an estimation function 444 which may correspond to a program in the memory 41.

This estimation function 444 estimates collection conditions for X-ray images to be acquired, so that the first trained model Md1 will serve as an adapted model. In this relation, the first feature amounts of the training data include first X-ray conditions and first imaging conditions as the collection conditions for the input images used in the training data. The second feature amount of an X-ray image includes second X-ray conditions and second imaging conditions as the collection conditions for this X-ray image.

Assuming that the second X-ray conditions deviate from the first X-ray conditions at a first deviation degree and the second imaging conditions deviate from the first imaging conditions at a second deviation degree, the estimation function 444 estimates collection conditions for X-ray images to be acquired in the subsequent operations, in such a manner that either the second X-ray conditions with the first deviation degree or the second imaging conditions with the second deviation degree, whichever have a larger deviation degree than the other, are replaced with the collection conditions for an intended input image that correspond to the conditions with the larger deviation degree.

Other configurations are the same as those in the first embodiment.

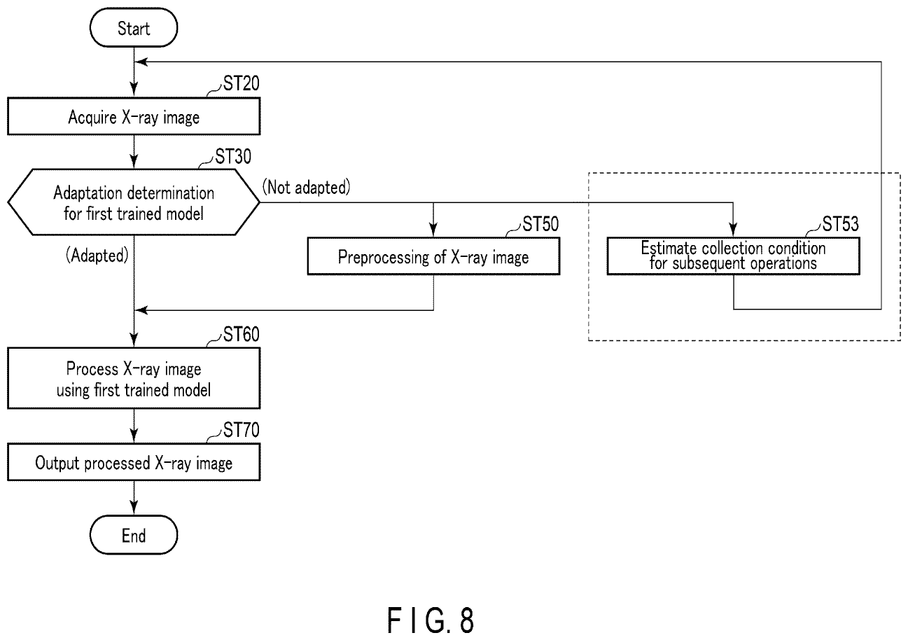

Next, operations of the X-ray diagnostic apparatus 1 configured as above will be described with reference to the flowchart in FIG. 8.

Suppose that the operations in steps ST20 to ST30 have been performed in the same manner as described above. If the result of the determination in step ST30 indicates that the first trained model Md1 is adapted, operations in step ST60 and onward are performed in the same manner as described above. If the result of the determination in step ST30 indicates that the first trained model Md1 is not adapted, the above described operations in step ST50 to ST70 are performed. Here, an operation in step ST53 is performed in parallel with the operation in step ST50.

In step ST53, the processing circuitry 44 estimates collection conditions for X-ray images to be acquired, so that the first trained model Md1 will serve as an adapted model. In one example, the processing circuitry 44 calculates a first deviation degree indicating a degree of deviation of the second X-ray conditions from the first X-ray conditions and a second deviation degree indicating a degree of deviation of the second imaging conditions from the first imaging conditions. Specifically, the processing circuitry 44 calculates, in a manner similar to the above, the first deviation degree by dividing a difference between the second X-ray conditions x and the center of gravity (average value μ) of the distribution of the first X-ray conditions by the standard deviation σ of the first X-ray conditions. Also, the processing circuitry 44 calculates the second deviation degree by dividing a difference between the second imaging conditions x and the center of gravity (average value μ) of the distribution of the first imaging conditions by the standard deviation σ of the first imaging conditions.

The processing circuitry 44 estimates collection conditions for X-ray images to be acquired in the subsequent operations, in such a manner that either the second X-ray conditions with the first deviation degree or the second imaging conditions with the second deviation degree, whichever have a larger deviation degree than the other, are replaced with the collection conditions for an intended input image that correspond to the conditions with the larger deviation degree. For example, if the first deviation degree is larger, the processing circuitry 44 estimates collection conditions for X-ray images to be acquired in the subsequent operations, in such a manner that the second X-ray conditions with the first deviation degree are replaced with the first X-ray conditions related to the first deviation degree. On the other hand, if the second deviation degree is larger, the processing circuitry 44 estimates collection conditions for X-ray images to be acquired in the subsequent operations, in such a manner that the second imaging conditions with the second deviation degree are replaced with the first imaging conditions related to the second deviation degree. Accordingly, the X-ray images in the subsequent operations are acquired by the imaging unit 10 and stored in the memory 41 based on the estimated collection conditions.

The processing circuitry 44 then performs operations in steps ST20 to ST70 for the X-ray images in the subsequent operations. Since the X-ray images in the subsequent operations are an outcome of the use of the first X-ray conditions or the first imaging conditions, which also constituted the collection conditions for the input images in the training data, it can be expected that the result of the determination in step ST30 indicates the first trained model MD1 to be an adapted model.

According to the second embodiment as described above, the processing circuitry 44 estimates collection conditions for X-ray images to be acquired, so that the first trained model Md1 will serve as an adapted model. Therefore, in addition to the advantages described above, adaptation of the first trained model Md1 to X-ray images acquired based on the estimated collection conditions can be attained.

Also according to the second embodiment, the first feature amounts include first X-ray conditions and first imaging conditions as collection conditions for the input images used in the training data. The second feature amount includes second X-ray conditions and second imaging conditions as collection conditions for an X-ray image. Assuming that the second X-ray conditions deviate from the first X-ray conditions at a first deviation degree and the second imaging conditions deviate from the first imaging conditions at a second deviation degree, the processing circuitry 44 estimates collection conditions for X-ray images to be acquired in the subsequent operations, in such a manner that either the second X-ray conditions with the first deviation degree or the second imaging conditions with the second deviation degree, whichever have a larger deviation degree than the other, are replaced with the collection conditions for an intended input image that correspond to the conditions with the larger deviation degree. Therefore, in addition to the advantages described above, adaptation of the first trained model Md1 to X-ray images acquired in subsequent operations based on the estimated collection conditions can be attained.

### (Modifications of Second Embodiment)

The description of the second embodiment has assumed the use of a relationship in size between the first deviation degree and the second deviation degree, but the embodiment is not limited to this. For example, a relationship in size between a weighted first deviation degree and a weighted second deviation degree which are obtained by multiplying the X-ray conditions and the imaging conditions by respective weighting coefficients according to their importance levels may instead or additionally be used. According to such a modification, the collection conditions for X-ray images can be estimated according to importance levels of the X-ray conditions and the imaging conditions, and therefore, X-ray images to which adaptation is more likely to be established can be acquired.

### Third Embodiment

The third embodiment may be understood as a modification of the first embodiment. The third embodiment considers the possibility of failing the adaptation establishment by a single image processing (preprocessing), and adopts a configuration in which an X-ray image after the image processing is used for an adaptation determination.

Accordingly, the processing function 443 of the processing circuitry 44 conducts a loop process of repeating the above determination after the image processing (preprocessing) for a given number of times, or a given length of time, equal to or below a threshold. More specifically, the threshold for the loop process is set according to a time required for the loop process, a prescribed output time, etc., from the viewpoint that the loop processes should be terminated if the preprocessing is not capable of solving the problem or if the prescribed output time is approaching.

The processing function 443 also subjects the X-ray image to, if the result of the last determination in the loop processes is “No”, alternative processing which differs from the image processing (preprocessing) and which does not use the first trained model Md1. The alternative processing here refers to processing for obtaining an X-ray image comparable to an X-ray image generated by the first trained model Md1, without using the first trained model Md1. More specifically, as the alternative processing, image processing or the like other than the machine learning-based technique may be discretionarily adopted. For example, if a given image processing applied by the first trained model Md1 is noise reduction, the alternative processing is noise reducing image processing, and if a given image processing applied by the first trained model Md1 is segmentation, the alternative processing is segmenting image processing. In other words, the alternative processing may be an originally intended image processing which is performed without the machine learning-based technique.

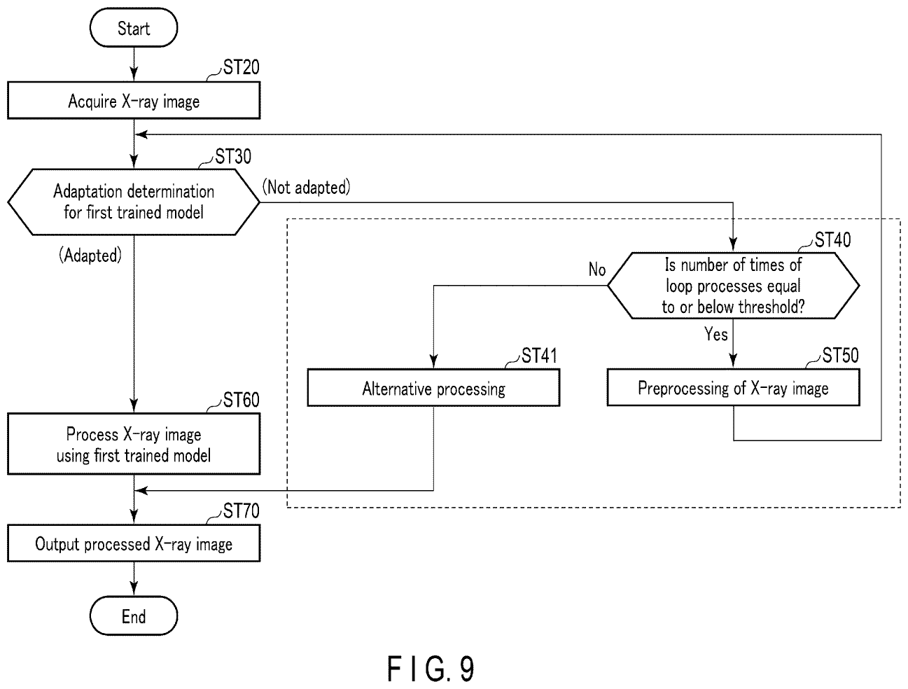

Next, operations of the X-ray diagnostic apparatus 1 configured as above will be described with reference to the flowchart in FIG. 9.

Suppose that the operations in steps ST20 to ST30 have been performed in the same manner as described above. If the result of the determination in step ST30 indicates that the first trained model Md1 is adapted, operations in step ST60 and onward are performed in the same manner as described above. If the result of the determination in step ST30 indicates that the first trained model Md1 is not adapted, an operation in step ST40 is performed before transition to step ST40 described above.

In step ST40, the processing circuitry 44 determines whether or not the number of times of the performed loop processes of repeating the determination after the image processing is equal to or below the threshold, so that the number of times of the loop processes will be kept equal to or below the threshold. Note that, for the operations shown in FIG. 9, the number of times of the loop process is incremented by one from initial value zero each time a set of steps ST50 and ST30 is carried out. If it is determined in step ST40 that the number of times of the performed loop process is equal to or below the threshold, another loop process is conducted in which the image processing (preprocessing) in step ST50 is performed and then the operation flow transitions to step ST30. The image processing (preprocessing) in step ST50 is applied to the X-ray image as many times as the number of loop processes performed. If, on the other hand, the result of the last determination in step ST40 in the loop processes is “No”, the operation flow transitions to step ST41.

In step ST41, the processing circuitry 44 subjects the X-ray image to alternative processing different from the image processing in step ST50 and performed without use of the first trained model Md1. After step ST41, the operation in step ST70 is performed in the same manner as described above.

According to the third embodiment as described above, the processing circuitry 44 conducts a loop process of repeating the determination after the image processing for a number of times equal to or below a threshold. As such, in addition to the advantages described above, the loop processes can be terminated upon the number of the performed loop processes exceeding the threshold, and therefore, an excessive repeat of the loop process can be avoided while the image processing (preprocessing) is showing no effect.

According to the third embodiment, further, the processing circuitry 44 subjects the X-ray image to, if the result of the last determination in the loop processes is “No”, alternative processing which differs from the image processing (preprocessing) and which does not use the first trained model Md1. Thus, in addition to the advantages described above, it is possible, with this configuration of conducting alternative processing after terminating the loop, to utilize the alternative processing to make up for the failure of the repeated image processing in establishing the adaptation of the first trained model Md1.

### (Modifications of Third Embodiment)

The description of the third embodiment has assumed a determination as to whether or not the number of times of the loop processes is equal to or below a threshold, but the embodiment is not limited to this. For example, whether or not the length of time of the loop processes is equal to or below a threshold may be determined. Such a modification can also provide the same effects and advantages as described for the third embodiment.

The description of the third embodiment has assumed as many applications of the image processing (preprocessing) to the X-ray image in loop, as the number of loop processes performed. However, the embodiment is not limited to this. For example, an initially acquired X-ray image may be subjected to the image processing (preprocessing) again in each loop process, with the intensity of correction suitably adjusted. Such a modification can also provide the same effects and advantages as described for the third embodiment.

### Fourth Embodiment

The fourth embodiment may be understood as a modification of the first embodiment. The fourth embodiment adopts a configuration in which the first trained model Md1 is prepared so that it will serve as an adapted model.

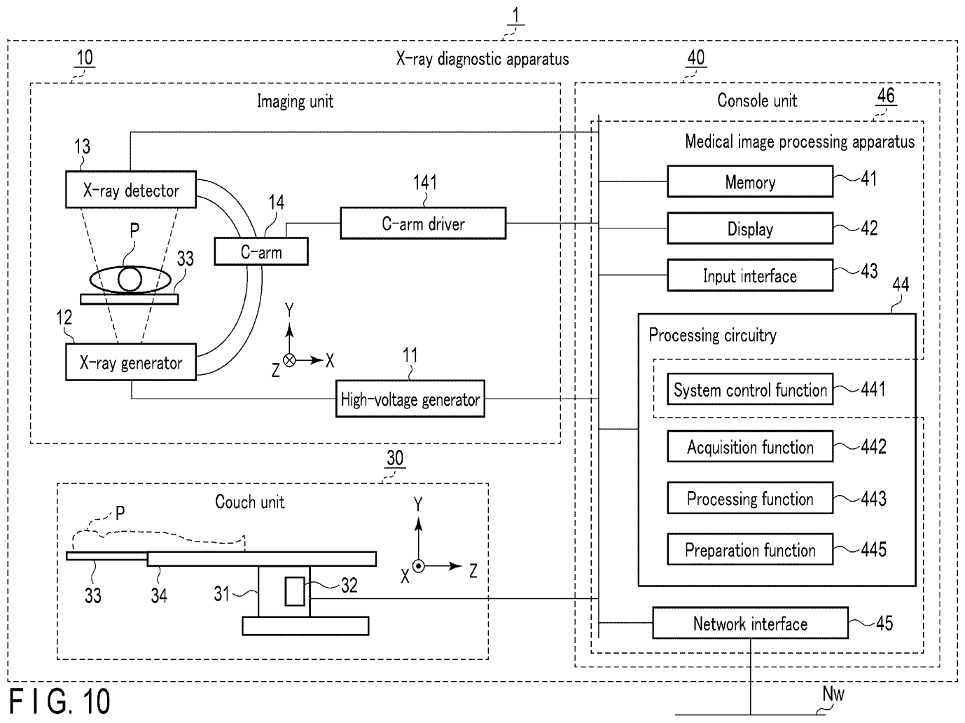

Accordingly, the processing circuitry 44 further realizes, as shown in FIG. 10, a preparation function 445 which may correspond to a program in the memory 41.

The preparation function 445 here prepares the first trained model so that it will serve as an adapted model. For example, as the preparation, the preparation function 445 conducts retraining of the machine learning model Md while the image processing (preprocessing) is being carried out, and upon finishing the retraining, updates the existing first trained model Md1 to the new first trained model Md1 obtained by the retraining.

More specifically, and for example, the preparation function 445 retrains the machine learning model Md in the memory 41 based on training data involving input images similar to the X-ray image to which the existing first trained model Md1 is not adapted, so as to generate the new first trained model Md1 as a trained machine learning model and store it in the memory 41.

Other configurations are the same as those in the first embodiment.

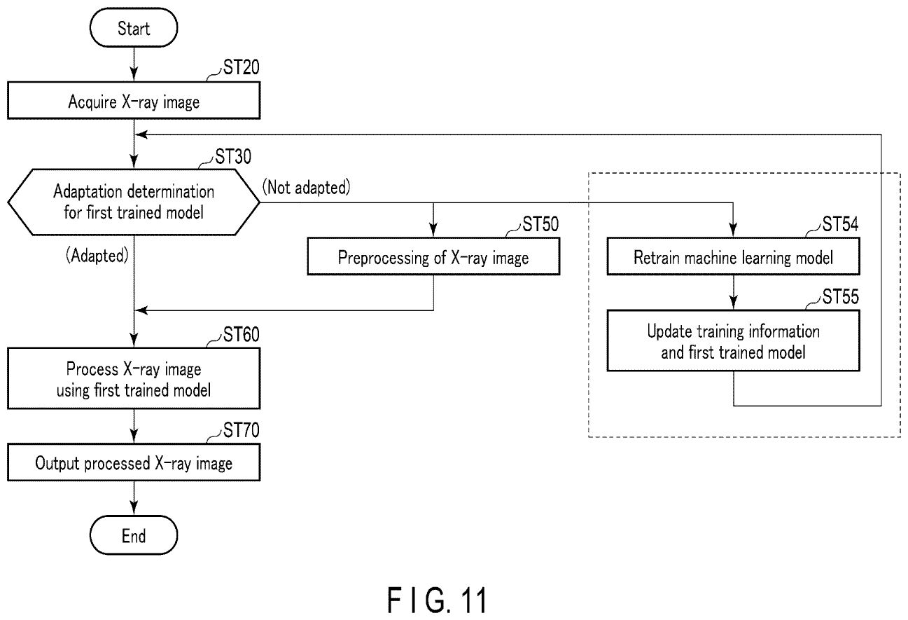

Next, operations of the X-ray diagnostic apparatus 1 configured as above will be described with reference to the flowchart in FIG. 11.

Suppose that the operations in steps ST20 to ST30 have been performed in the same manner as described above. If the result of the determination in step ST30 indicates that the first trained model Md1 is adapted, operations in step ST60 and onward are performed in the same manner as described above. If the result of the determination in step ST30 indicates that the first trained model Md1 is not adapted, the above described operations in steps ST50 to ST70 are performed. Here, an operation in step ST54 is performed in parallel with the operation in step ST50.

In step ST54, the processing circuitry 44 prepares the first trained model Md1 so that it will serve as an adapted model. For example, the processing circuitry 44 conducts retraining of the machine learning model Md in parallel with the image processing (preprocessing) in step ST50. As the training data for use in the retraining, for example, input images having first feature amounts close to the second feature amount of the X-ray image are employed from among the multiple training data pieces. Accordingly, in terms of what is shown in FIG. 6, the training data that has a distribution in which an average value μ of the first feature amounts is shifted toward the second feature amount x is used for the retraining. Note that the number of pieces of the training data showing the shifted average value μ of the first feature amounts is less than that without the shift. That is, the training data for use in the retraining has a distribution showing a smaller lobe shape with an average value μ of the first feature amounts shifted toward the second feature amount x as compared to what is shown in FIG. 6. Step ST54 is thus finished.

In step ST55 after the retraining, the processing circuitry 44 performs the preparation by updating the existing first trained model Md1 to the new first trained model Md1 obtained by the retraining. In this updating, the processing circuitry 44 also updates the training information 41a for the new first trained model Md1. For an X-ray image acquired after finishing step ST55, the adaptation determination in step ST30 is performed based on the updated training information 41a. If the result of the determination indicates that the updated first trained model Md1 is adapted, the operation in step ST60 is performed using the updated first trained model Md1. After step ST60, the operation in step ST70 is performed in the same manner as described above.

According to the fourth embodiment as described above, the processing circuitry 44 prepares the first trained model Md1 so that it will serve as an adapted model. Therefore, in addition to the advantages described above, a first trained model Md1 exhibiting a good adaptation can be prepared separately from image processing on the X-ray image.

Also according to the fourth embodiment, the processing circuitry 44 performs the preparation by retraining the machine learning model in parallel with the image processing, and upon finishing the retraining, updating the existing first trained model Md1 to the new first trained model Md1 obtained by the retraining. Therefore, in addition to the advantages described above, a first trained model exhibiting an even greater adaptation to the X-ray image can be prepared by the retraining operation in parallel with the image processing on the X-ray image.

### (Modifications of Fourth Embodiment)

While the description of the fourth embodiment has assumed retraining of one machine learning model Md, the embodiment is not limited to this. For example, multiple machine learning models Md may be retrained. By way of example, in instances where a training process is time-consuming, multiple machine learning models Md may be retrained using multiple pieces of sampled training data, so as to prepare multiple trained models in advance. Subsequently, if adaptation failure is determined, one of the first trained models Md1 that has been retrained using multiple pieces of the training data having first feature amounts close to the second feature amount of the X-ray image is selected and used. Such a modification can also provide the same effects and advantages as described for the fourth embodiment.

### Fifth Embodiment

The fifth embodiment may be understood as a modification of the first embodiment. The fifth embodiment adopts a configuration in which the first trained model Md1 is prepared so that it will serve as an adapted model. The fifth embodiment differs from the fourth embodiment in that it prepares the adapted first trained model Md1 in advance. Accordingly, the processing circuitry 44 further realizes, as shown in FIG. 10, the preparation function 445 which may correspond to a program in the memory 41.

Here, the processing function 443 of the processing circuitry 44 determines whether or not each of multiple first trained models Md1 is adapted to an acquired X-ray image.

If one or more of the multiple first trained models Md1 are determined to be adapted, the preparation function 445 selects the most adapted first trained model Md1 for the preparation. Note that not all training data is used but the training data with a limited range of feature amounts is used to prepare the multiple first trained models Md1. In one example, the multiple first trained models Md1 are prepared through machine learning processes using training data related to X-ray conditions, namely, tube voltage values of around 60 kVp, 80 kVp, and 100 kVp, respectively. Then, the adaptation determination is performed so that the most adapted first trained model Md1 is selected for the preparation.

Other configurations are the same as those in the first embodiment.

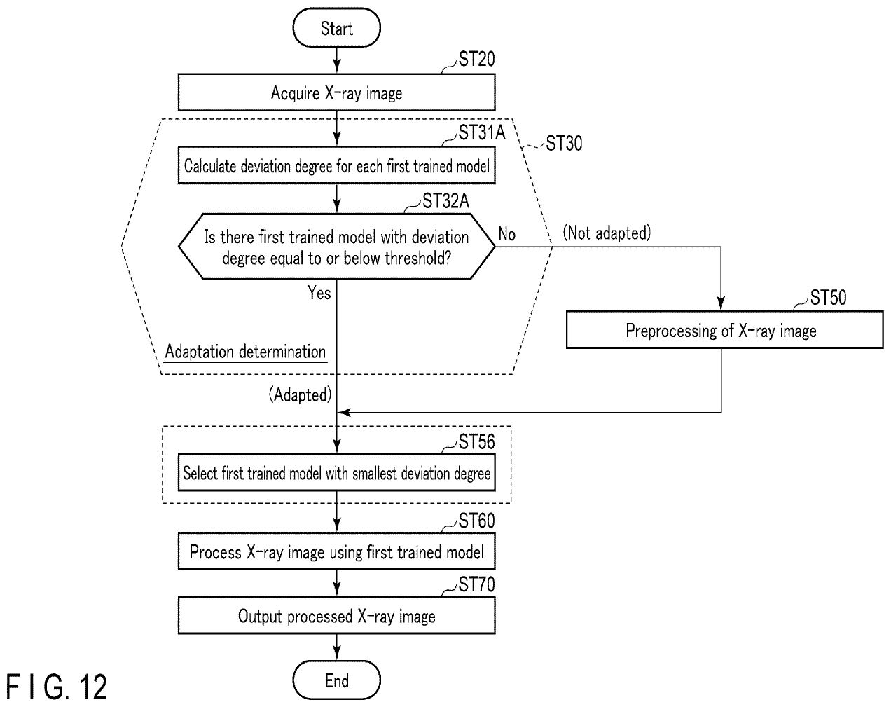

Next, operations of the X-ray diagnostic apparatus 1 configured as above will be described with reference to the flowchart in FIG. 12.

Suppose that the operation in step ST20 has been performed in the same manner as described above, and the operation in step ST30 is started.

In step ST30, the processing circuitry 44 determines whether or not each of multiple first trained models Md1 is adapted to the acquired X-ray image. This step ST30 for adaptation determination is constituted by steps ST31A to ST32A.

For example, in step ST31A, the processing circuitry 44 calculates a deviation degree for each of the multiple first trained models Md1.

Subsequently, in step ST32A, the processing circuitry 44 performs the determination according to each of the calculated deviation degrees. In one example, the processing circuitry 44 determines the adaptation or non-adaptation according to whether or not there is a first trained model Md1 with a deviation degree equal to or below the threshold among the multiple first trained models Md1 with their respective deviation degrees. If the result of the determination is “No”, the above described operations in steps ST50 to ST70 are performed. On the other hand, if the result of the determination in step ST32A indicates that one or more of the multiple first trained models Md1 are adapted, the operation flow transitions to step ST56.

In step ST56, the processing circuitry 44 selects, for the preparation, the most adapted first trained model Md1 from among the one or more first trained models Md1 determined to be adapted in step ST32A. Here, the most adapted first trained model Md1 may be, for example, the first trained model Md1 having the smallest deviation degree.

After step ST56, the operations in step ST60 and onward are performed in the same manner as described above.

According to the fifth embodiment as described above, the processing circuitry 44 prepares the first trained model Md1 so that it will serve as an adapted model. Therefore, in addition to the advantages described above, a first trained model Md1 exhibiting a good adaptation can be prepared in advance.

Also according to the fifth embodiment, the processing circuitry 44 determines whether or not each of multiple first trained models Md1 is adapted to the acquired X-ray image. If one or more of the multiple first trained models Md1 are determined to be adapted, the processing circuitry 44 selects the most adapted first trained model Md1 for the preparation. Therefore, in addition to the advantages described above, a first trained model Md1 exhibiting a good adaptation can be prepared from among multiple first trained models Md1 prior to image processing on the X-ray image. Moreover, since the first trained model Md1 specialized in handling the X-ray image can be selectively used, an even more precise outcome of the given image processing by the first trained model Md1 can be expected.

### Sixth Embodiment

The sixth embodiment may be understood as a modification of the first embodiment. The sixth embodiment adopts a configuration in which conditions for initial collection are estimated so that an X-ray image to which the first trained model Md1 is adapted will be acquired.

Accordingly, the processing circuitry 44 further realizes, as shown in FIG. 7, an estimation function 444 which may correspond to a program in the memory 41.

This estimation function 444 estimates collection conditions for an X-ray image to be acquired, so that the first trained model Md1 will serve as an adapted model. In this relation, the first feature amounts of the training data include first X-ray conditions and first imaging conditions as the collection conditions for the input images used in the training data. The second feature amount of an X-ray image includes second X-ray conditions and second imaging conditions as the collection conditions for this X-ray image.

Assuming that the second X-ray conditions deviate from the first X-ray conditions at a first deviation degree and the second imaging conditions deviate from the first imaging conditions at a second deviation degree, the estimation function 444 estimates collection conditions for an X-ray image to be initially acquired, in such a manner that either the second X-ray conditions with the first deviation degree or the second imaging conditions with the second deviation degree, whichever have a larger deviation degree than the other, are replaced with the collection conditions for an intended input image that correspond to the conditions with the larger deviation degree.

Other configurations are the same as those in the first embodiment.

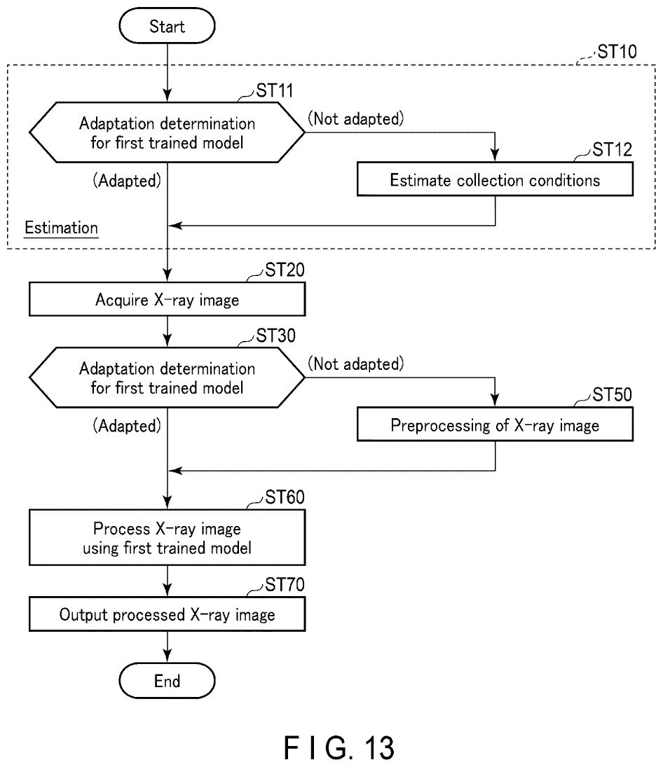

Next, operations of the X-ray diagnostic apparatus 1 configured as above will be described with reference to the flowchart in FIG. 13.

As a first step, the processing circuitry 44 in step ST10 estimates collection conditions for an X-ray image to be acquired, so that the first trained model Md1 will serve as an adapted model. This step ST10 for estimation is constituted by steps ST11 to ST12.

In step ST11, for example, the processing circuitry 44 determines whether or not the first trained model Md1, to which an X-ray image is to be input, is adapted to the X-ray image. In one example, the processing circuitry 44 acquires a deviation degree by calculation, and determines the adaptation or non-adaptation according to whether or not the acquired deviation degree is equal to or below a threshold as in step ST30. Note, however, that the second feature amount of the X-ray image used for the calculation of a deviation degree here does not include an image feature amount, since an actual X-ray image has not been acquired yet. The second feature amount of the X-ray image, used for the deviation degree calculation, includes second X-ray conditions and second imaging conditions as the collection conditions for this X-ray image. The first feature amounts of the training data, used for the deviation degree calculation, include first X-ray conditions and first imaging conditions as the collection conditions for the input images used in the training data. Accordingly, the processing circuitry 44 at least calculates a first deviation degree indicating a degree of deviation of the second X-ray conditions from the first X-ray conditions and a second deviation degree indicating a degree of deviation of the second imaging conditions from the first imaging conditions. If the result of the determination in step ST11 is “No”, that is, if the deviation degree D exceeds the threshold, the operation flow transitions to step ST12. If the deviation degree is equal to or below the threshold, and thus, the determination result is that the first trained model Md1 is adapted, the operation flow transitions to step ST20.

In step ST12, the processing circuitry 44 selects a larger one of the first deviation degree and the second deviation degree. The processing circuitry 44 estimates collection conditions for an X-ray image to be initially acquired, in such a manner that either the second X-ray conditions or the second imaging conditions that involve the selected deviation degree are replaced with the collection conditions for an intended input image that correspond to the conditions with the selected deviation degree. Step ST12 is thus finished, and the operation flow transitions to step ST20.

In step ST20, an initial X-ray image is acquired by the imaging unit 10 and stored in the memory 41 based on the estimated collection conditions. Accordingly, the processing circuitry 44 acquires the X-ray image stored in the memory 41.

Subsequently, the operations in step ST30 and onward are performed in the same manner as described above. Note that, for the operation in step ST30, the second feature amount of the X-ray image used for the deviation degree calculation includes an image feature amount, since the actual X-ray image has been acquired.

According to the sixth embodiment as described above, the processing circuitry 44 estimates collection conditions for an X-ray image to be acquired, so that the first trained model Md1 will serve as an adapted model. Therefore, in addition to the advantages described above, adaptation of the first trained model Md1 to an X-ray image acquired based on the estimated collection conditions can be attained.

Also according to the sixth embodiment, the first feature amounts include first X-ray conditions and first imaging conditions as collection conditions for the input images used in the training data. The second feature amount includes second X-ray conditions and second imaging conditions as collection conditions for an X-ray image. Assuming that the second X-ray conditions deviate from the first X-ray conditions at a first deviation degree and the second imaging conditions deviate from the first imaging conditions at a second deviation degree, the processing circuitry 44 estimates collection conditions for an X-ray image to be initially acquired, in such a manner that either the second X-ray conditions with the first deviation degree or the second imaging conditions with the second deviation degree, whichever have a larger deviation degree than the other, are replaced with the collection conditions for an intended input image that correspond to the conditions with the larger deviation degree. Therefore, in addition to the advantages described above, adaptation of the first trained model Md1 to an X-ray image initially acquired based on the estimated collection conditions can be attained.

Moreover, a modification similar to the modification of the second embodiment is applicable to the sixth embodiment so that the same effects and advantages can be obtained.

### Seventh Embodiment

The seventh embodiment may be understood as a modification of each of the first to sixth embodiments. The seventh embodiment adopts a configuration in which image processing as the preprocessing is performed by a second trained model. To avoid redundant explanations, the description will use a representative example where this seventh embodiment is applied to the first embodiment.

The processing function 443 of the processing circuitry 44 here additionally uses a second trained model if the result of the above determination is “No” (that is, the first trained model Md1 is not adapted to an acquired X-ray image). The second trained model has been trained to generate an input image used in the training data for the first trained model Md1, based on a variant image corresponding to the input image with varied image feature amounts. In other words, the processing function 443 inputs the X-ray image to the second trained model so as to perform image processing of generating an X-ray image having the image feature amounts of the input X-ray image approximated to those of the input images used in the training data.

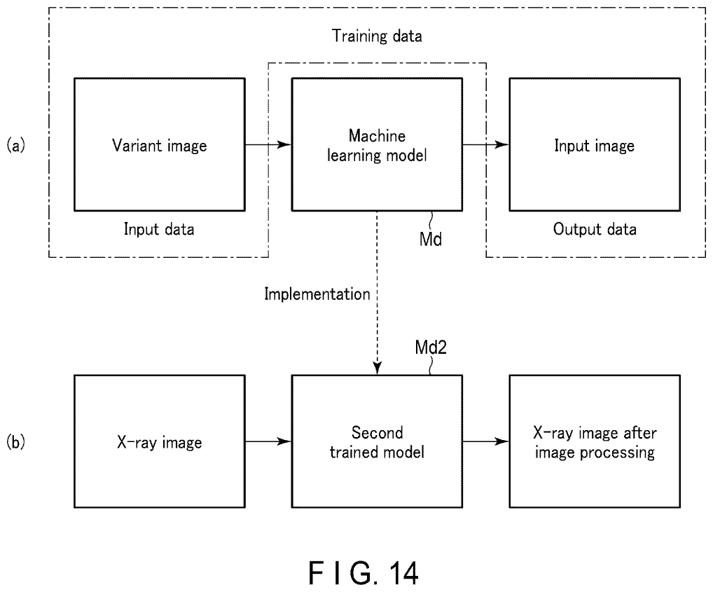

The second trained model is an already-trained model Md2 which has been obtained by having a machine learning model Md undergo a machine learning process with training data including input data and output data and which is set in the memory 41. See FIG. 14(a) illustrating a training instance. The training data for the second trained model Md2 includes a set of input data and output data, where the input data is variant images of which distribution of image feature amounts is varied from that of the input images used in the training data for the first trained model Md1, and the output data is the input image without the distribution variation. The variant image is prepared as, for example, an image corresponding to the input image with a “variation” added. The “variation” is given on condition that it is not covered by the range of training data for the first trained model Md1. The training data for the second trained model Md2 is employed in the machine learning process for the capability of generating an input image to which the first trained model Md1 is adapted based on a variant image to which the first trained model Md1 is not adapted. The machine learning process employs multiple pieces of the training data. Also, the second trained model Md2 corresponds to the trained machine learning model Md in implementation, and upon receiving an input of the acquired X-ray image, it outputs the X-ray image to which the image processing has been applied. See FIG. 14(b) illustrating a use instance. Note that, for implementation, the second trained model Md2 may be set in the memory 41 in advance, before shipment of the X-ray diagnostic apparatus 1 from the factory. As another option, the trained machine learning model Md may be acquired from a server device or the like (not shown in the figure) and installed in the memory 41 for implementation of the second trained model Md2, after the factory shipment of the X-ray diagnostic apparatus 1. These implementation manners are also applicable to the other embodiments and modifications.

The remaining configurations are the same as those in the first embodiment.

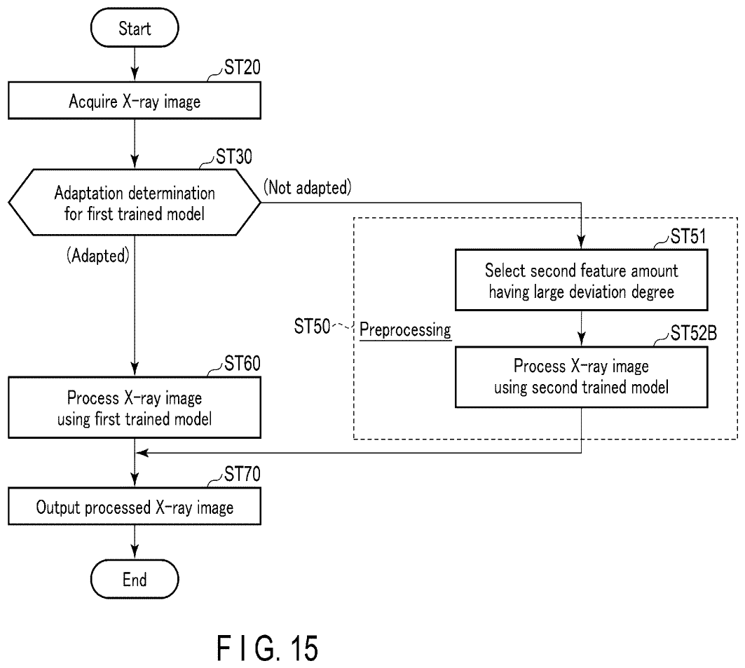

Next, operations of the X-ray diagnostic apparatus 1 configured as above will be described with reference to the flowchart in FIG. 15.

Suppose that the operations in steps ST20 to ST30 have been performed in the same manner as described above. If the result of the determination in step ST30 indicates that the first trained model Md1 is adapted, operations in step ST60 and onward are performed in the same manner as described above. If the result of the determination in step ST30 indicates that the first trained model Md1 is not adapted, the above described operations in steps ST50 to ST70 are performed. Here, step ST50 includes preprocessing performed through steps ST51 and ST52B.

In step ST51, the processing circuitry 44 selects the second feature amount having a deviation degree greater than the threshold. In step ST52B, the processing circuitry 44 inputs the X-ray image to the second trained model Md2, trained to generate an input image used in the training data for the first trained model Md1 based on the variant image of which image feature amounts are varied from the input image. The processing circuitry 44 thereby conducts image processing of generating an X-ray image corresponding to the input X-ray image in which image feature amounts have been approximated to the input images used in the training data for the first trained model Md1. In this manner, step ST50 constituted by steps ST51 and ST52B is finished. After step ST50, the operations in step ST60 and onward are performed in the same manner as described above.

According to the seventh embodiment as described above, the processing circuitry 44 inputs an X-ray image to the second trained model Md2, trained to generate an input image used in the training data for the first trained model Md1 based on a variant image of which distribution of image feature amounts is varied from that of the input image. The processing circuitry 44 thereby conducts image processing of generating an X-ray image corresponding to the input X-ray image with a distribution of image feature amounts approximated to that of the input images used in the training data for the first trained model Md1. Therefore, in addition to the advantages described above, image processing, i.e., preprocessing, can also be implemented through the machine learning technique by the configuration of subjecting X-ray images to the image processing using the second trained model Md2.

### Eighth Embodiment

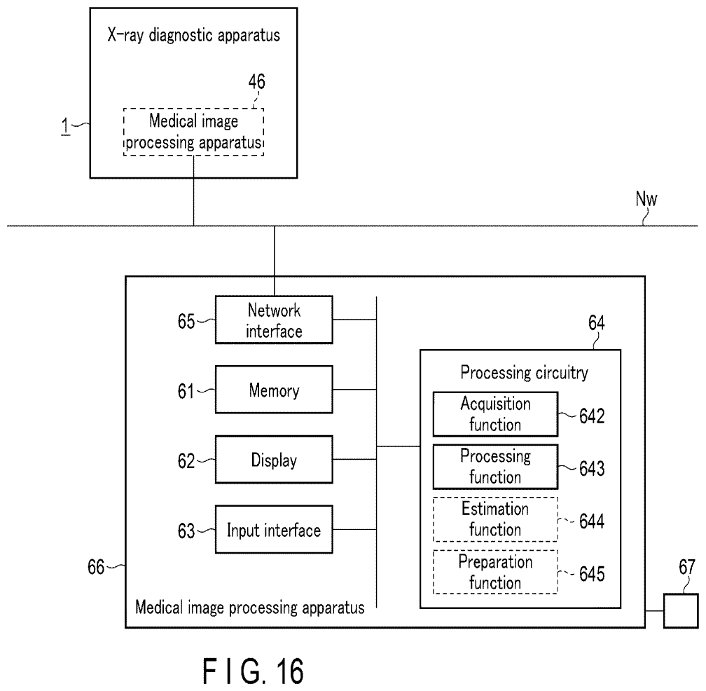

The eighth embodiment may be understood as a modification of each of the first to seventh embodiments. The eighth embodiment encompasses a medical image processing apparatus 66 which is an external apparatus to the X-ray diagnostic apparatus 1 as shown in FIG. 16. The medical image processing apparatus 66 includes at least one memory 61, a display 62, an input interface 63, processing circuitry 64, and a network interface 65. The processing circuitry 64 includes a processor and a memory (which are not shown in the figure). The processor of the processing circuitry 64 reads and executes a program or programs in the memory 61 for realizing corresponding functions including an acquisition function 642, a processing function 643, an estimation function 644, and a preparation function 645. In one example, such a program or programs are intended to be preliminarily installed into a computer or a workstation, etc. from a network or a non-transitory computer-readable storage medium 67, and be executed by a processor of the computer, etc. so that the computer is caused to implement the functions of the medical image processing apparatus 66. Note that at least one of the estimation function 644 and the preparation function 645 is not essential and may be provided as an optional feature.

The medical image processing apparatus 66 can communicate with the X-ray diagnostic apparatus 1 via the network Nw, and it has the same functions as those of the medical image processing apparatus 46. As such, the foregoing descriptions of the medical image processing apparatus 46 of the X-ray diagnostic apparatus 1, if the most significant digit “4” of each reference numeral is replaced with “6”, can apply to the medical image processing apparatus 66, and redundant descriptions will be omitted. Each of the medical image processing apparatus 46 and the medical image processing apparatus 66 is an example of a medical image processing apparatus performing a medical image processing method.

More specifically, the memory 61 has a similar configuration to that of the memory 41 described above, and it stores X-ray images sequentially generated by the X-ray diagnostic apparatus 1 which performs sequential generation of X-ray images of a subject. The generated X-ray images are sent by the X-ray diagnostic apparatus 1, received by the network interface 65, and written in the memory 61. The memory 61 also stores one or more programs for the medical image processing apparatus 66. Each of the memory 61 and the memory 41 is an example of a storage.

The display 62 has a similar configuration to that of the display 42 described above, and it displays X-ray images, etc., under the control of the processing circuitry 44.

The input interface 63 has a similar configuration to that of the input interface 43 described above, and it outputs electric signals corresponding to input operations by an operator to the processing circuitry 64.

The processing circuitry 64 has a similar configuration to that of the processing circuitry 44 described above, and it includes a processor and a memory (not shown) so that various types of information entered or set via the input interface 63 are stored in the memory. The processor of the processing circuitry 64 reads and executes a program or programs in the memory 61 for realizing corresponding functions including the acquisition function 642, the processing function 643, the estimation function 644, and the preparation function 645. The processing circuitry 64 with the acquisition function 642 is an example of an acquirer. The processing circuitry 64 with the processing function 643 is an example of a processor. The processing circuitry 64 with the estimation function 644 is an example of an estimator. The processing circuitry 64 with the preparation function 645 is an example of a preparator.

The acquisition function 642 acquires X-ray images from the X-ray diagnostic apparatus 1 performing sequential generation of X-ray images of a subject and stores the acquired X-ray images in the memory 61. The acquisition function 642 is a function equivalent to the above described acquisition function 442.

The processing function 643 determines whether or not the first trained model, to which an X-ray image is to be input, is adapted to this X-ray image, and if not, applies image processing to the X-ray image so that the first trained model will serve as an adapted model. The processing function 643 is a function equivalent to the above described processing function 443.

The estimation function 644 is a function equivalent to the above described estimation function 444, and it estimates collection conditions for X-ray images so that the first trained model will serve as an adapted model. Accordingly, the acquisition function 642 acquires X-ray images based on the estimated collection conditions.

The preparation function 645 is a function equivalent to the above described preparation function 445, and it prepares the first trained model so that the first trained model will serve as an adapted model.

The network interface 65 has a similar configuration to that of the network interface 45 described above, and it is a circuitry component for realizing communications with external devices through cables or wirelessly, or through the combination of cable and wireless means. The external devices here include, for example, the X-ray diagnostic apparatus 1 operating as a modality.

The medical image processing apparatus 66 as described above functions as an entity equivalent to the medical image processing apparatus 46 of the X-ray diagnostic apparatus 1 provided as an external device.

According to the eighth embodiment as described above, the processing circuitry 64 in the medical image processing apparatus 66 acquires an X-ray image. The processing circuitry 64 determines whether or not the first trained model, to which the X-ray image is to be input, is adapted to this X-ray image, and if not, applies image processing to the X-ray image so that the first trained model will serve as an adapted model. Therefore, an adaptation of the trained model to the X-ray image can be attained. That is, this medical image processing apparatus can also realize the same effects and advantages as those according to the first to seventh embodiments. Moreover, such effects and advantages are also attainable from the configuration in which a program or programs corresponding to the functions of the medical image processing apparatus are mounted on a computer.

According to at least one embodiment described above, the adaptation of a trained model to X-ray images can be attained.

The terminology “processor” used herein refers to, for example, a central processing unit (CPU) or a graphics processing unit (GPU), or various types of circuitry components which may be an application-specific integrated circuit (ASIC), a programmable logic device (such as a simple programmable logic device (SPLD), a complex programmable logic device (CPLD), or a field programmable gate array (FPGA)), and so on. The processor reads and executes a program or programs stored in the memory to realize intended functions. If, for example, the processor is a CPU, the processor reads and executes the program or programs stored in the memory to realize the functions. If, for example, the processor is an ASIC, the functions are directly incorporated into circuitry of the processor in the form of a logic circuit, instead of corresponding programs being stored in the memory. Note that each processor in the embodiments, etc. is not limited to a single circuit-type processor, and multiple independent circuits may be combined as one processor to realize the intended functions. Furthermore, multiple components or features as given in FIG. 1, 7, 10, or 16 may be integrated as one processor to realize their functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments may be made without departing from the spirit of the inventions. The accompanying claims and their equivalents are intended to cover such forms or modifications as would fall within the scope and spirit of the inventions.

1. An X-ray diagnostic apparatus comprising processing circuitry configured to:
acquire an X-ray image; and
perform a determination as to whether or not a first trained model, to which the X-ray image is to be input, is adapted to the X-ray image, and if the determination indicates that the first trained model is not adapted to the X-ray image, apply image processing to the X-ray image so that the first trained model serves as an adapted model.

2. The X-ray diagnostic apparatus according to claim 1, wherein
training data for the first trained model comprises first feature amounts and the X-ray image comprises a second feature amount, and
the processing circuitry is configured to perform the determination based on a degree of deviation of the second feature amount from a center of gravity of a distribution of the first feature amounts.

3. The X-ray diagnostic apparatus according to claim 2, further comprising a memory for storing the center of gravity and a standard deviation of the distribution of the first feature amounts,
wherein the processing circuitry is configured to acquire the degree of deviation by dividing a difference between the second feature amount and the center of gravity by the standard deviation, and to perform the determination according to whether or not the degree of deviation is equal to or below a threshold.

4. The X-ray diagnostic apparatus according to claim 2, wherein the processing circuitry is configured to, if the determination indicates that the first trained model is not adapted to the X-ray image, select the second feature amount having the degree of deviation greater than a threshold so as to conduct the image processing associated with the selected second feature amount.

5. The X-ray diagnostic apparatus according to claim 4, wherein the processing circuitry is configured to set an average value of the distribution of image feature amounts among the first feature amounts to be a target value of the image processing, and to apply the image processing to the X-ray image based on the target value.

6. The X-ray diagnostic apparatus according to claim 1, wherein the processing circuitry is further configured to estimate a collection condition for an X-ray image to be acquired, so that the first trained model serves as the adapted model.

7. The X-ray diagnostic apparatus according to claim 2, wherein the processing circuitry is further configured to estimate a collection condition for an X-ray image to be acquired, so that the first trained model serves as the adapted model.

8. The X-ray diagnostic apparatus according to claim 7, wherein
the processing circuitry is configured to, if the determination indicates that the first trained model is not adapted to the X-ray image, select the second feature amount having the degree of deviation greater than a threshold so as to conduct the image processing associated with the selected second feature amount,
the first feature amounts comprise a first X-ray condition and a first imaging condition as a collection condition for an input image used in the training data,
the second feature amount comprises a second X-ray condition and a second imaging condition as a collection condition for the X-ray image, and
the processing circuitry is configured to, assuming that the second X-ray condition deviates from the first X-ray condition at a first deviation degree and the second imaging condition deviates from the first imaging condition at a second deviation degree, estimate a collection condition for an X-ray image to be acquired in a subsequent operation, in such a manner that either the second X-ray condition with the first deviation degree or the second imaging condition with the second deviation degree, whichever has a larger deviation degree, is replaced with the collection condition for the input image that corresponds to the condition with the larger deviation degree.

9. The X-ray diagnostic apparatus according to claim 1, wherein the processing circuitry is configured to conduct one or more loop processes of repeating the determination after the image processing for a number of times, or a length of time, equal to or below a threshold.

10. The X-ray diagnostic apparatus according to claim 9, wherein the processing circuitry is configured to, if the determination performed last time in the one or more loop processes indicates that the first trained model is not adapted to the X-ray image, subject the X-ray image to alternative processing which differs from the image processing and which does not use the first trained model.

11. The X-ray diagnostic apparatus according to claim 1, wherein the processing circuitry is further configured to perform preparation of the first trained model so that the first trained model serves as the adapted model.

12. The X-ray diagnostic apparatus according to claim 11, wherein the processing circuitry is configured to perform the preparation by retraining a machine learning model in parallel with the image processing, and upon finishing the retraining, updating the first trained model to a new first trained model obtained by the retraining.

13. The X-ray diagnostic apparatus according to claim 11, wherein
the first trained model is more than one, and
the processing circuitry is configured to
determine whether or not each of the more than one first trained model is adapted to the X-ray image, and
if one or more of the more than one first trained model are determined to be adapted, select a most adapted one of the more than one first trained model for the preparation.

14. The X-ray diagnostic apparatus according to claim 7, wherein
the processing circuitry is configured to, if the determination indicates that the first trained model is not adapted to the X-ray image, select the second feature amount having the degree of deviation greater than a threshold so as to conduct the image processing associated with the selected second feature amount,
the first feature amounts comprise a first X-ray condition and a first imaging condition as a collection condition for an input image used in the training data,
the second feature amount comprises a second X-ray condition and a second imaging condition as a collection condition for the X-ray image which is to be acquired, and
the processing circuitry is configured to, assuming that the second X-ray condition deviates from the first X-ray condition at a first deviation degree and the second imaging condition deviates from the first imaging condition at a second deviation degree, estimate a collection condition for the X-ray image to be initially acquired, in such a manner that either the second X-ray condition with the first deviation degree or the second imaging condition with the second deviation degree, whichever has a larger deviation degree, is replaced with the collection condition for the input image that corresponds to the condition with the larger deviation degree.

15. The X-ray diagnostic apparatus according to claim 1, wherein the processing circuitry is configured to, if the determination indicates that the first trained model is not adapted to the X-ray image, input the X-ray image to a second trained model so as to conduct the image processing to generate an X-ray image corresponding to the input X-ray image with a distribution of image feature amounts approximated to that of input images used in training data for the first trained model,
the second trained model being trained to generate an input image used in the training data for the first trained model Md1 based on a variant image of which distribution of image feature amounts is varied from that of the input image.

16. A medical image processing apparatus comprising processing circuitry configured to:
acquire an X-ray image; and
perform a determination as to whether or not a first trained model, to which the X-ray image is to be input, is adapted to the X-ray image, and if the determination indicates that the first trained model is not adapted to the X-ray image, apply image processing to the X-ray image so that the first trained model serves as an adapted model.

17. A medical image processing method comprising:
acquiring an X-ray image; and
performing a determination as to whether or not a first trained model, to which the X-ray image is to be input, is adapted to the X-ray image, and if the determination indicates that the first trained model is not adapted to the X-ray image, applying image processing to the X-ray image so that the first trained model serves as an adapted model.
